(19) United States
(12) Patent Application Publication
(10) Pub. No.: US 2011/0053223 A1
(43) Pub. Date: Mar. 3, 2011

(54) CELL CULTURE METHODS TO MAKE ANTIBODIES WITH ENHANCED ADCC FUNCTION

(76) Inventors: Robert Bayer, San Diego, CA (US); Feng Li, Mission Viejo, CA (US); Efren Pacis, Oceanside, CA (US); Marcella Yu, Medford, MA (US)

(21) Appl. No.: 12/852,389

(22) Filed: Aug. 6, 2010

Related U.S. Application Data
(60) Provisional application No. 61/234,238, filed on Aug. 14, 2009.

(51) Int. Cl. C12P 21/06

(52) U.S. Cl. 435/69.6

(57) ABSTRACT

The present invention concerns antibodies with enhanced antibody-dependent cell mediated cytotoxicity (ADCC) and method for preparation thereof.

### FIELD OF THE INVENTION

The present invention concerns antibodies enhanced antibody-dependent cell mediated cytotoxicity (ADCC) and method for preparation thereof.

### BACKGROUND OF THE INVENTION

Antibody-dependent cell-mediated cytotoxicity (ADCC) is a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. It is known that among antibodies of the human IgG class, the IgG1 subclass has the highest ADCC activity and CDC activity, and currently most of the humanized antibodies in clinical oncological practice, including commercially available HERCEPTIN® (trastuzumab) and RITUXAN® (rituximab), which require high effector functions for the expression of their effects, are antibodies of the human IgG1 subclass.

In order to enhance the potency of therapeutic antibodies, it is often desirable to modify the antibodies with respect to effector function, e.g., so as to enhance antigen-dependent cell-mediated cyotoxicity (ADCC) and/or complement dependent cytotoxicity (CDC) of the antibody. This can be of particular benefit in the oncology field, where therapeutic monoclonal antibodies bind to specific antigens on tumor cells and induce an immune response resulting in destruction of the tumor cell. By enhancing the interaction of IgG with killer cells bearing Fc receptors, these therapeutic antibodies can be made more potent.

Enhancement of effector functions, such as ADCC, may be achieved by various means, including introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

Another approach to enhance the effector function of antibodies, including antibodies of the IgG class, is to engineer the glycosylation pattern of the antibody Fc region. An IgG molecule contains an N-linked oligosaccharide covalently attached at the conserved Asn297 of each of the CH2 domains in the Fc region. The oligosaccharides found in the Fc region of serum IgGs are mostly biantennary glycans of the complex type. A number of antibody glycoforms have been reported as having a positive impact on antibody effector function, including antibody-dependent cell mediated cytotoxicity (ADCC). Thus, glycoengineering of the carbohydrate component of the Fc-part, particularly reducing core fucosylation, has been reported by Shinkawa T, et al., J Biol Chem. 2003; 278:3466-73; Niwa R, et al., Cancer Res 2004; 64:2127-33; Okazaki A, et al., J Mol Biol 2004; 336:1239-49; and Shields R L, et al., J Biol Chem 2002; 277:26733-40.

Antibodies with select glycoforms have been made by a number of means, including the use of glycosylation pathway inhibitors, mutant cell lines that have absent or reduced activity of particular enzymes in the glycosylation pathway, engineered cells with gene expression in the glycosylation pathway either enhanced or knocked out, and in vitro remodeling with glycosidases and glycosyltransferases. Rothman et al., 1989; Molecular Immunology 26: 1113-1123, expressed monoclonal IgG in the presence of the glucosidase inhibitors castanospermine and N-methyldeoxynojirimycin, and the mannosidase I inhibitor deoxymannojirimycin. Umana et al., Nature Biotechnology 1999; 17: 176-180, describe enhanced effector function of a chimeric IgG1 expressed in a CHO cell line expressing GNT-III. Shields et al., 2002; JBC 277:26733-26740, 2002, describe enhanced ADCC in human IgG1 expressed in the Lec13 cell line, which is deficient in its ability to add fucose. Shinkawa et al., 2003; JBC 278: 3466-3473, 2003, showed that an anti-CD20 IgG1 expressed in YB2/0 cells showed more than 50-fold higher ADCC using purified human peripheral blood mononuclear cells as effector than those produced by Chinese hamster ovary (CHO) cell lines. Monosaccharide composition and oligosaccharide profiling analysis showed that low fucose (Fuc) content of complex-type oligosaccharides was characteristic in YB2/0-produced IgG1s compared with high Fuc content of CHO-produced IgG1s. Kanda et al., 2006; Glycobiology 17, 104-118, describe enhanced ADCC in rituximab bearing afucosyl complex, afucosyl hybrid, Man5, and Man8,9 glycans. Yamane-Ohnuki et al., Biotechnol Bioeng 2004; 87:614-22, achieved a reduction of core fucosylation by recombinant antibody expression in CHO cells lacking core-fucosyl transferase activity, whereas Mori et al., Biotechnol Bioeng 2004; 88:901-8, maximized effector functions of expressed antibodies using fucosyl transferase specific short interfering RNA (siRNA).

Compared to main antibody glycoforms (G0, G1, and G2), antibodies with high mannose glycoforms (Man5-9) are present at low levels, typically below 2-5%. Some recent publications indicate that antibodies bearing predominantly the Man5 glycoform have some unique properties in terms of potency, immunogenicity and clearance rate. Antibodies bearing predominantly the Man5 glycoform have been described by Wright and Morrison; 1994, J. Exp. Med. 180:1087-1096; 1998; J. Immunology 160: 3393-3402). The antibodies were expressed in the lec1 cell line, which does not have an active GlcNAc Transferase I. Judging from the biphasic clearance curve in FIG. 8 of the J. Exp. Med. paper, there appears to be at least two distinct populations of antibody with different clearance characteristics. The more rapidly cleared population of IgG is presumably antibody bearing Man7,8,9 glycoforms.

As described above, the glycosylation pattern of recombinant antibodies is essential for full biological activity in vivo. However, the antibody glycosylation structure often varies with the change of cell culture conditions. Critical parameters are associated with cell line, culture media and process conditions. The changes of mAb glycosylation profile during development and manufacturing often raise some concerns about product quality and comparability. Therefore, it is highly desirable to understand how those key process parameters and media components impact mAb glycosylation during the cell culture production process. Other approaches to select glycoforms have utilized cell culturing methods aimed at controlling or reducing the mannose content of recombinantly produced proteins or peptides (Wu et al.; US Publication 2007/0190057) or producing a particular glycoprotein with occupancy of a specific glycosylation site (tissue plasminogen activator; Andersen et al.; U.S. Pat. No. 6,506,598).

### SUMMARY OF THE INVENTION

The present application illustrates the effects of cell culture process conditions on Man5 antibody levels. Three culture conditions were found to have significant impacts. Manipulating these conditions, including media osmolality, culture duration and media Manganese concentration, can increase or decrease Man5 levels, and therefore enable better control for product glycosylation during the cell culture process.

In one aspect, the present invention concerns a method for making an antibody or a fragment thereof, or an immunoadhesin or a fragment thereof, bearing Man5 glycans, comprising culturing a mammalian cell line engineered to express an antibody or a fragment thereof, or an immunoadhesin or a fragment thereof, under conditions suitable for the accumulation of Man5 glycoproteins for a duration of time greater than 12 days.

In another aspect, the present invention concerns a method for making an antibody or a fragment thereof, or an immunoadhesin or a fragment thereof, bearing Man5 glycans, comprising culturing a mammalian cell line engineered to express an antibody or a fragment thereof, or an immunoadhesin or a fragment thereof, in culture medium having a basal media osmolality of about 300 mOsm/Kg or greater.

In yet another aspect, the present invention concerns a method for making an antibody or a fragment thereof, or an immunoadhesin or a fragment thereof, bearing Man5 glycans, comprising culturing a mammalian cell line engineered to express an antibody or a fragment thereof, or an immunoadhesin or a fragment thereof, in culture medium having a Manganese concentration of about 0.25 micromolar or less.

In a further aspect, the present invention concerns a method for recombinant production of an antibody, an immunoadhesin, or a fragment thereof with about 10% to 30% Man5 glycans in the carbohydrate structure thereof, comprising expressing nucleic acid encoding said antibody or antibody fragment in a mammalian cell line, wherein said fragment comprises at least one glycosylation site. Followed by culturing said cell line under conditions suitable for the accumulation of Man5 glycoproteins for a duration of time greater than 12 days, and isolating said antibody or a fragment thereof, or an immunoadhesin or a fragment thereof, bearing predominantly Man5 glycans.

In another aspect, the present invention concerns a method for recombinant production of an antibody, an immunoadhesin, or a fragment thereof with about 10% to 30% Man5 glycans in the carbohydrate structure thereof, comprising expressing nucleic acid encoding said antibody or antibody fragment in a mammalian cell line, wherein said fragment comprises at least one glycosylation site. Followed by culturing said cell line in culture medium having a basal media osmolality of about 300 mOsm/Kg or greater, and isolating said antibody or a fragment thereof, or an immunoadhesin or a fragment thereof, bearing predominantly Man5 glycans.

In a still further aspect, the present invention concerns a method for recombinant production of an antibody, an immunoadhesin, or a fragment thereof with about 10% to 30% Man5 glycans in the carbohydrate structure thereof, comprising expressing nucleic acid encoding said antibody or antibody fragment in a mammalian cell line, wherein said fragment comprises at least one glycosylation site. Followed by culturing said cell line in culture medium having a Manganese concentration of about 0.25 micromolar or less, and isolating said antibody or a fragment thereof, or an immunoadhesin or a fragment thereof, bearing predominantly Man5 glycans.

In various embodiments of the present invention, the above cell culturing methods may be used alone or combination to achieve maximal Man5 levels.

In additional embodiments of the present invention, the above cell culturing methods may be used alone or combination with other methods to achieve maximal Man5 levels.

In a particular embodiment, the cell culturing methods of the present invention may be used in conjunction with a mammalian cell lacking GlcNAc Transferase I activity.

In all aspects, the mammalian cell line may, for example, be a Chinese Hamster Ovary (CHO) cell line.

In all aspects, the cell lines and methods of the present invention can be used for the production of any antibody, including, without limitation, antibodies of diagnostic or therapeutic interest, such as, antibodies binding to one or more of the following antigens: CD3, CD4, CD8, CD19, CD20, CD22, CD34, CD40, EGF receptor (EGFR, HER1, ErbB1), HER2 (ErbB2), HER3 (ErbB3), HER4 (ErbB4), macrophage receptor (CRIg), tumor necrosis factors, TRAIL/Apo-2, LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DRS, EGFL7, neuropilins and receptors, netrins and receptors, slit and receptors, sema and receptors, semaphorins and receptors, robo and receptors, and M1.

The antibodies and antibody fragments may be chimeric or humanized, and specifically include chimeric and humanized anti-CD20 antibodies, where, in a specific embodiment, the antibody is rituximab or ocrelizumab.

In another embodiment, the humanized antibody is an anti-HER2, anti-HER1, anti-VEGF or anti-IgE antibody, including, without limitation, trastuzumab, pertuzumab, bevacizumab, ranibizumab, and omalizumab, as well as fragments, variants and derivatives of such antibodies.

Antibody fragments include, for example, complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide, provided that they are glycosylated.

In particular embodiments, the invention concerns a method for making an antibody or a fragment thereof, or an immunoadhesin or a fragment thereof, bearing 10% or greater, or 20% or greater, or 25% or greater, or 30% or greater, or 35% or greater, or 40% or greater, or 45% or greater, or 50% or greater, or 55% or greater, or 60% or greater, or 65% or greater, or 70% or greater, or 75% or greater Man5 glycans, comprising culturing a mammalian cell line according to the above embodiments under conditions such that said antibody or a fragment thereof, or an immunoadhesin or a fragment thereof is produced, wherein said fragment comprises at least one glycosylation site.

### BRIEF DESCRIPTION OF THE DRAWINGS

FIG. 1. Man5 level at various days of culture duration. The Man5 level was determined by CE-glycan assay, and the errors bars represent standard deviations.

FIG. 2. Comparison of Man5 level after 22 days culture. Four different osmolality in basal media was tested (300, 330, 360, 400 mOsm) in conjunction with two different feed media osmolality levels (750 and 1250). The Man5 level was determined by CE-glycan assay.

FIG. 3A. Man5 levels measured after adding different amounts (0.25, 0.5 or 1 μM) of MnCl₂ to the culture medium on day 3. The Man5 level was determined by CE-glycan assay.

FIG. 3B. Man5 level with the addition of MnCl₂ (1 μM) on various days of a total 14 day culture. The Man5 level was determined by CE-glycan assay.

### DETAILED DESCRIPTION OF THE INVENTION

### I. Definitions

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)) and mediates slower catabolism, thus longer half-life.

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of immunoglobulins are called a, d, e, g, and m, respectively. The subunit structures and three-dimensional configurations of different classes of immunoglobulins are well known and described generally in, for example, Abbas et al., Cellular and Mol. Immunology, 4th ed. (2000). An antibody may be part of a larger fusion molecule, formed by covalent or non-covalent association of the antibody with one or more other proteins or peptides.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., BioTechnology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994). The humanized antibody includes a Primatized™ antibody wherein the antigen-binding region of the antibody is derived from an antibody produced by immunizing macaque monkeys with the antigen of interest.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs/HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al., Bio/Technology 10:779-783 (1992) describes affinity maturation by V_{H} and V_{L} domain shuffling. Random mutagenesis of CDR/HVR and/or framework residues is described by: Barbas et al., Proc Nat. Acad. Sci. USA 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al., J. Mol. Biol. 226:889-896 (1992).

The term “polyclonal antibody” is used to refer to a population of antibody molecules synthesized by a population of B cells.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise an antigen binding arm linked to an Fc sequence capable of conferring in vivo stability to the fragment. Examples of antibody fragments include, but are not limited to, Fab, Fab′, F(ab)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide. Specifically within the scope of the invention are bispecific antibody fragments.

Antibodies are glycoproteins, with glycosylation in the Fc region. Thus, for example, the Fc region of an IgG immunoglobulin is a homodimer comprising interchain disulfide-bonded hinge regions, glycosylated CH2 domains bearing N-linked oligosaccharides at asparagine 297 (Asn-297), and non-covalently paired CH3 domains. Glycosylation plays an important role in effector mechanisms mediated FcγRI, FcγRII, FcγRIII, and Clq. Thus, antibody fragments of the present invention must include a glycosylated Fc region and an antigen-binding region.

The terms “bispecific antibody” and “bispecific antibody fragment” are used herein to refer to antibodies or antibody fragments with binding specificity for at least two targets. If desired, multi-specificity can be combined by multi-valency in order to produce multivalent bispecific antibodies that possess more than one binding site for each of their targets. For example, by dimerizing two scFv fusions via the helix-turn-helix motif, (scFv)₁-hinge-helix-turn-helix-(scFv)₂, a tetravalent bispecific miniantibody was produced (Muller et al., FEBS Lett. 432(1-2):45-9 (1998)). The so-called ‘ di-bi-miniantibody’ possesses two binding sites to each of it target antigens.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, Vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO93/1161; Hudson et al., (2003) Nat. Med. 9:129-134; and Hollinger et al., Proc. Natl. Acad. Sci. USA 90: 6444-6448 (1993). Triabodies and tetrabodies are also described in Hudson et al., (2003) Nat. Med. 9:129-134.

A “naked antibody” is an antibody (as herein defined) that is not conjugated to a heterologous molecule, such as a cytotoxic moiety or radiolabel.

The term “therapeutic antibody” refers to an antibody that is used in the treatment of disease. A therapeutic antibody may have various mechanisms of action. A therapeutic antibody may bind and neutralize the normal function of a target associated with an antigen. For example, a monoclonal antibody that blocks the activity of the of protein needed for the survival of a cancer cell causes the cell's death. Another therapeutic monoclonal antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen.

The antibody may bind to any protein, including, without limitation, a member of the HER receptor family, such as HER1 (EGFR), HER2, HER3 and HER4; CD proteins such as CD3, CD4, CD8, CD19, CD20, CD21, CD22, and CD34; cell adhesion molecules such as LFA-1, Mol, p150,95, VLA-4, ICAM-1, VCAM and av/p3 integrin including either α or β or subunits thereof (e.g. anti-CD11a, anti-CD18 or anti-CD11b antibodies); macrophage receptor such as CRIg, tumor necrosis factors such as TRAIL/Apo-2, growth factors such as vascular endothelial growth factor (VEGF); IgE; blood group antigens; flk2/flt3 receptor; obesity (OB) receptor; and protein C. Other exemplary proteins include growth hormone (GH), including human growth hormone (hGH) and bovine growth hormone (bGH); growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, factor, tissue factor, and von Willebrands factor; anti-clotting factors such as Protein C; atrial natriuretic factor; lung surfactant; a plasminogen activator, such as urokinase or tissue-type plasminogen activator (t-PA); bombazine; thrombin; tumor necrosis factor-α and -β; enkephalinase; RANTES (regulated on activation normally T-cell expressed and secreted); human macrophage inflammatory protein (MIP-1-α); serum albumin such as human serum albumin (HSA); mullerian-inhibiting substance; relaxin A-chain; relaxin B-chain; prorelaxin; mouse gonadotropin-associated peptide; DNase; inhibin; activin; receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins (IGFBPs); erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides. Many other antibodies and/or other proteins may be used in accordance with the instant invention, and the above lists are not meant to be limiting.

A “biologically functional fragment” of an antibody comprises only a portion of an intact antibody, wherein the portion retains at least one, and as many as most or all, of the functions normally associated with that portion when present in an intact antibody. In one embodiment, a biologically functional fragment of an antibody comprises an antigen binding site of the intact antibody and thus retains the ability to bind antigen. In another embodiment, a biologically functional fragment of an antibody, for example one that comprises the Fc region, retains at least one of the biological functions normally associated with the Fc region when present in an intact antibody, such as FcRn binding, antibody half life modulation, ADCC function and complement binding. In one embodiment, a biologically functional fragment of an antibody is a monovalent antibody that has an in vivo half life substantially similar to an intact antibody. For example, such a biologically functional fragment of an antibody may comprise an antigen binding arm linked to an Fc sequence capable of conferring in vivo stability to the fragment. As used herein, the term “immunoadhesin” designates antibody-like molecules which combine the “binding domain” of a heterologous protein (an “adhesin”, e.g. a receptor, ligand or enzyme) with the effector functions of immunoglobulin constant domains. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG1, IgG.2, IgG3, or IgG4 subtypes, IgA, IgE, IgD or IgM. For further details of immunoadhesins, ligand binding domains and receptor binding domains see, e.g. U.S. Pat. Nos. 5,116,964; 5,714,147; and 6,406,604, the disclosures of which are hereby expressly incorporated by reference.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. In preferred embodiments, the antibody will be purified to greater than 95% by weight of antibody as determined by non-reducing SDS-PAGE, CE-SDS, or Bioanalyzer. Isolated antibody includes the antibody in situ within recombinant cells since at least one component of the antibody's natural environment will not be present. Ordinarily, however, isolated antibody will be prepared by at least one purification step.

“Purified” means that a molecule is present in a sample at a concentration of at least 80-90% by weight of the sample in which it is contained.

The protein, including antibodies, which is purified is preferably essentially pure and desirably essentially homogeneous (i.e. free from contaminating proteins etc.).

An “essentially pure” protein means a protein composition comprising at least about 90% by weight of the protein, based on total weight of the composition, preferably at least about 95% by weight.

An “essentially homogeneous” protein means a protein composition comprising at least about 99% by weight of protein, based on total weight of the composition.

The terms “Protein A” and “ProA” are used interchangeably herein and encompasses

Protein A recovered from a native source thereof, Protein A produced synthetically (e.g. by peptide synthesis or by recombinant techniques), and variants thereof which retain the ability to bind proteins which have a C_{H}2/C_{H}3 region, such as an Fc region. Protein A can be purchased commercially from Repligen, GE Healthcare and Fermatech. Protein A is generally immobilized on a solid phase support material. The term “ProA” also refers to an affinity chromatography resin or column containing chromatographic solid support matrix to which is covalently attached Protein A.

The term “chromatography” refers to the process by which a solute of interest in a mixture is separated from other solutes in a mixture as a result of differences in rates at which the individual solutes of the mixture migrate through a stationary medium under the influence of a moving phase, or in bind and elute processes.

The term “affinity chromatography” and “protein affinity chromatography” are used interchangeably herein and refer to a protein separation technique in which a protein of interest or antibody of interest is reversibly and specifically bound to a biospecific ligand. Preferably, the biospecific ligand is covalently attached to a chromatographic solid phase material and is accessible to the protein of interest in solution as the solution contacts the chromatographic solid phase material. The protein of interest (e.g., antibody, enzyme, or receptor protein) retains its specific binding affinity for the biospecific ligand (antigen, substrate, cofactor, or hormone, for example) during the chromatographic steps, while other solutes and/or proteins in the mixture do not bind appreciably or specifically to the ligand. Binding of the protein of interest to the immobilized ligand allows contaminating proteins or protein impurities to be passed through the chromatographic medium while the protein of interest remains specifically bound to the immobilized ligand on the solid phase material. The specifically bound protein of interest is then removed in active form from the immobilized ligand with low pH, high pH, high salt, competing ligand, and the like, and passed through the chromatographic column with the elution buffer, free of the contaminating proteins or protein impurities that were earlier allowed to pass through the column. Any component can be used as a ligand for purifying its respective specific binding protein, e.g. antibody.

The terms “non-affinity chromatography” and “non-affinity purification” refer to a purification process in which affinity chromatography is not utilized. Non-affinity chromatography includes chromatographic techniques that rely on non-specific interactions between a molecule of interest (such as a protein, e.g. antibody) and a solid phase matrix.

A “cation exchange resin” refers to a solid phase which is negatively charged, and which thus has free cations for exchange with cations in an aqueous solution passed over or through the solid phase. A negatively charged ligand attached to the solid phase to form the cation exchange resin may, e.g., be a carboxylate or sulfonate. Commercially available cation exchange resins include carboxy-methyl-cellulose, sulphopropyl (SP) immobilized on agarose (e.g. SP-SEPHAROSE FAST FLOW™ or SP-SEPHAROSE HIGH PERFORMANCE™, from GE Healthcare) and sulphonyl immobilized on agarose (e.g. S-SEPHAROSE FAST FLOW™ from GE Healthcare). A “mixed mode ion exchange resin” refers to a solid phase which is covalently modified with cationic, anionic, and hydrophobic moieties. A commercially available mixed mode ion exchange resin is BAKERBOND ABX™ (J.T. Baker, Phillipsburg, N.J.) containing weak cation exchange groups, a low concentration of anion exchange groups, and hydrophobic ligands attached to a silica gel solid phase support matrix.

The term “anion exchange resin” is used herein to refer to a solid phase which is positively charged, e.g. having one or more positively charged ligands, such as quaternary amino groups, attached thereto. Commercially available anion exchange resins include DEAE cellulose, QAE SEPHADEX™ and FAST Q SEPHAROSE™ (GE Healthcare).

A “buffer” is a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., ed. Calbiochem Corporation (1975). In one embodiment, the buffer has a pH in the range from about 2 to about 9, alternatively from about 3 to about 8, alternatively from about 4 to about 7 alternatively from about 5 to about 7. Non-limiting examples of buffers that will control the pH in this range include MES, MOPS, MOPSO, Tris, HEPES, phosphate, acetate, citrate, succinate, and ammonium buffers, as well as combinations of these.

The “loading buffer” is that which is used to load the composition comprising the polypeptide molecule of interest and one or more impurities onto the ion exchange resin. The loading buffer has a conductivity and/or pH such that the polypeptide molecule of interest (and generally one or more impurities) is/are bound to the ion exchange resin or such that the protein of interest flows through the column while the impurities bind to the resin.

The “intermediate buffer” is used to elute one or more impurities from the ion exchange resin, prior to eluting the polypeptide molecule of interest. The conductivity and/or pH of the intermediate buffer is/are such that one or more impurity is eluted from the ion exchange resin, but not significant amounts of the polypeptide of interest.

The term “wash buffer” when used herein refers to a buffer used to wash or re-equilibrate the ion exchange resin, prior to eluting the polypeptide molecule of interest. Conveniently, the wash buffer and loading buffer may be the same, but this is not required.

The “elution buffer” is used to elute the polypeptide of interest from the solid phase. The conductivity and/or pH of the elution buffer is/are such that the polypeptide of interest is eluted from the ion exchange resin.

A “regeneration buffer” may be used to regenerate the ion exchange resin such that it can be re-used. The regeneration buffer has a conductivity and/or pH as required to remove substantially all impurities and the polypeptide of interest from the ion exchange resin.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein with regard to amounts or numerical values (and not as reference to the chemical process of reduction), denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and
where Y is the total number of amino acid residues in B.
   It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Percent (%) nucleic acid sequence identity” is defined as the percentage of nucleotides in a candidate sequence that are identical with the nucleotides in a reference Factor D-encoding sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Sequence identity is then calculated relative to the longer sequence, i.e. even if a shorter sequence shows 100% sequence identity with a portion of a longer sequence, the overall sequence identity will be less than 100%.

“Treatment” refers to both therapeutic treatment and prophylactic or preventative measures. Those in need of treatment include those already with the disorder as well as those in which the disorder is to be prevented. “Treatment” herein encompasses alleviation of the disease and of the signs and symptoms of the particular disease.

A “disorder” is any condition that would benefit from treatment with the antibody or immunoadhesin. This includes chronic and acute disorders or diseases including those pathological conditions which predispose the mammal to the disorder in question. Non-limiting examples of disorders to be treated herein include carcinomas and allergies.

“Mammal” for purposes of treatment refers to any animal classified as a mammal, including humans, non-human higher primates, other vertebrates, domestic and farm animals, and zoo, sports, or pet animals, such as dogs, horses, cats, cows, etc. Preferably, the mammal is human.

The term “glycoform” refers to any of several different forms of a glycoprotein (or other biological glycoside) having different saccharides attached, or having a different structure.

Carbohydrate moieties are described herein with reference to commonly used nomenclature for oligosaccharides. A review of carbohydrate chemistry which uses this nomenclature can be found, for example, in Hubbard and Ivatt, Ann. Rev. Biochem. 50:555-583 (1981). This nomenclature includes, for instance, Man, which represents mannose; GlcNAc, which represents 2-N-acetylglucosamine; Gal which represents galactose; and Glc, which represents glucose. “Man7,8,9”, “Man5, 6” and “Man5” glycans are used herein to refer to the number of mannose residues of the ManₓGlcNAc₂ moiety. Essentially, mannose substituents of the Man₉GlcNAc₂ moiety can be removed by α-mannosidase I to generate N-linked Man₅₋₉GlcNAc₂, all of which are commonly found on vertebrate glycoproteins.

The term “osmolality,” as used herein, refers to a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution (1 mOsm/kg H.sub.2O at 38.degree. C. is equivalent to an osmotic pressure of 19 mm Hg). As used herein, the abbreviation “mOsm” means “milliosmoles/kg solution.”

As used herein, the terms “cell culture medium” and “culture medium” refer to a nutrient solution used for growing mammalian cells that typically provides at least one component from one or more of the following categories: 1) an energy source, usually in the form of a carbohydrate such as, for example, glucose; 2) one or more of all essential amino acids, and usually the basic set of twenty amino acids plus cysteine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The nutrient solution may optionally be supplemented with additional components to optimize growth of cells.

“RNAi knockdown” is used herein to refer to RNA interference technology, which is a method for regulating gene expression. RNA interference molecules can bind to single-stranded mRNA molecules with a complementary sequence and repress translation of particular genes. The RNA can be introduced exogenously (small interfering RNA, or siRNA), or endogenously by RNA producing genes (micro RNA, or miRNA).

An “interfering RNA” or “small interfering RNA (siRNA)” is a double stranded RNA molecule less than about 30 nucleotides in length that reduces expression of a target gene. Interfering RNAs may be identified and synthesized using known methods (Shi Y., Trends in Genetics 19(1):9-12 (2003), WO/2003056012 and WO2003064621), and siRNA libraries are commercially available, for example from Dharmacon, Lafayette, Colo. Frequently, siRNAs can be successfully designed to target the 5′ end of a gene.

Alpha mannosidase I inhibitors are compounds that are capable of inhibiting alpha mannosidases, including inhibitors that block only alpha 1,2 mannosidases as well as inhibitors that, in addition, are capable of inhibiting other mannosidases as well. Kifunensine, produced by the actinomycete Kitasatosporia kifunense 9482, is an alkaloid, corresponding to a cyclic oxamide derivative of 1-amino mannojirimycin, that inhibits α-mannosidase and asparagine-linked oligosaccharide processing. (Iwami, M., et al., J. Antibiot., 40: 612, (1987); Chandrosekaran, S., et al., J. Biol. Chem., 269: 3356, (1994)) Thiosugar derivatives that are more potent than kifunensine have been described (Sivapriya et al, Bioorg Med Chem (2007) 15 (17): 5659-65). Other alpha mannosidase inhibitors include, but are not limited to, iminocyclitols (Butters et al, Glycoconj J. (2009) epub), 1-deoxymannojirimycin (Bischoff et al, J. Biol. Chem. (1986) 261:4766-4774), kifunensine analogues (Hering et al, J Org Chem (2005) 70: 9892-904), and D-Mannonolactam Amidrazone (Pan et al, J Biol Chem (1992) 267: 8313-8318).

### II. Detailed Description

The present invention provides a method for preparing antibodies and antibody-like molecules, such as Fc fusion proteins (immunoadhesins), bearing predominantly Man5 glycans, but with decreased amounts of Man7, Man8, and Man9, in a mammalian host cell, by manipulating cell culturing conditions for a recombinant mammalian host cell producing the antibody or antibody-like molecule.

General Methods for the Recombinant Production of Antibodies

The antibodies and other recombinant proteins herein can be produced by well known techniques of recombinant DNA technology. Thus, aside from the antibodies specifically identified herein, the skilled practitioner could generate antibodies directed against an antigen of interest, e.g., using the techniques described below.

The antibodies produced in accordance with the present invention are directed against an antigen of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or ligand such as a growth factor. Exemplary molecular targets for antibodies encompassed by the present invention include CD proteins such as CD3, CD4, CD8, CD19, CD20, CD22, CD34, CD40; members of the ErbB receptor family such as the EGF receptor (EGFR, HER1, ErbB1), HER2 (ErbB2), HER3 (ErbB3) or HER4 (ErbB4) receptor; macrophage receptors such as CRIg, tumor necrosis factors such as TRAIL/Apo-2, cell adhesion molecules such as LFA-1, Mac1, p150,95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; mpl receptor; CTLA-4; protein C, neutropilins and receptors, EGF-C, ephrins and receptors, netrins and receptors, slit and receptors, anti-M1, or any of the other antigens mentioned herein. Antigens to which the antibodies listed above bind are specifically included within the scope herein.

For recombinant production of the antibody, the nucleic acid encoding it may be isolated and inserted into a replicable vector for further cloning (amplification of the DNA) or for expression. In another embodiment, the antibody may be produced by homologous recombination, e.g. as described in U.S. Pat. No. 5,204,244, specifically incorporated herein by reference. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, e.g., as described in U.S. Pat. No. 5,534,615 issued Jul. 9, 1996 and specifically incorporated herein by reference.

The antibodies of the present invention must be glycosylated, and thus suitable host cells for cloning or expressing the DNA encoding antibody chains or other antibody-like molecules include mammalian host cells. Interest has been great in mammalian host cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with expression or cloning vectors for antibody production and cultured in conventional nutrient media modified as appropriate for inducing promoters, selecting transformants, or amplifying the genes encoding the desired sequences.

The mammalian host cells may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The antibody composition prepared from the cells can be purified using, for example, hydroxylapatite chromatography, ion exchange chromatography, gel electrophoresis, dialysis, and affinity chromatography, with affinity chromatography being the primary purification step. The suitability of protein A as an affinity ligand depends on the species and isotype of any immunoglobulin Fc domain that is present in the antibody. Protein A can be used to purify antibodies that are based on human γ1, human γ2, or human γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the BAKERBOND ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin, chromatofocusing, SDS-PAGE, hydrophobic interaction chromatography, and ammonium sulfate precipitation are also available depending on the antibody to be recovered.

Following any preliminary purification step(s), the mixture comprising the antibody of interest and contaminants may be subjected to additional purification steps to achieve the desired level of purity.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (Sims et al., J. Immunol., 151:2296 (1993)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to another approach described in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies are contemplated. For example, trispecific antibodies can be prepared. Tutt et al. J. Immunol. 147: 60 (1991).

Immunoadhesins

The simplest and most straightforward immunoadhesin design combines the binding domain(s) of the adhesin (e.g. the extracellular domain (ECD) of a receptor) with the hinge and Fc regions of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc domain of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Just as the antibodies and antibody fragments, the immunoadhesin structures of the present invention must have an Fc region. Various exemplary assembled immunoadhesins within the scope herein are schematically diagrammed below:

AC_{H}-(AC_{H}, AC_{L}-AC_{H}, AC_{L}-V_{H}C_{H}, or V_{L}C_{L}-AC_{H});

AC_{L}-AC_{H}-(AC_{L}-AC_{H}, AC_{L}-V_{H}C_{H}, V_{L}C_{L}-AC_{H}, or V_{L}C_{L}-V_{H}C_{H})

AC_{L}-V_{H}C_{H}-(AC_{H}, or AC_{L}-V_{H}C_{H}, or V_{L}C_{L}-AC_{H});

V_{L}C_{L}-AC_{H}-(AC_{L}-V_{H}C_{H}, or V_{L}C_{L}-AC_{H}); and

(A-Y)ₙ-(V_{L}C_{L}-V_{H}C_{H})₂,

wherein each A represents identical or different adhesin amino acid sequences;

V_{L} is an immunoglobulin light chain variable domain;

V_{H} is an immunoglobulin heavy chain variable domain;

C_{L} is an immunoglobulin light chain constant domain;

C_{H} is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown.

However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom, et al., Mol. Immunol. 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell 61:1303-1313 (1990); and Stamenkovic et al., Cell 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Antibodies with Enhanced ADCC Function

Following the expression of proteins in eukaryotic, e.g. mammalian host cells, the proteins undergo post-translational modifications, often including the enzymatic addition of sugar residues, generally referred to as “glycosylation”.

Glycosylation of polypeptides is typically either N-linked or O-linked. N-linked refers to the attachment of the carbohydrate moiety to the side-chain of an asparagine residue. The tripeptide sequences, asparagine (Asn)-X-serine (Ser) and asparagine (Asn)-X-threonine (Thr), wherein X is any amino acid except proline, are recognition sequences for enzymatic attachment of the carbohydrate moiety to the asparagine side chain. O-linked glycosylation refers to the attachment of one of the sugars N-acetylgalactosamine, galactose, fucose, N-acetylglucosamine, or xylose to a hydroxyamino acid, most commonly serine or threonine, although 5-hydroxyproline or 5-hydroxylysine may also be involved in O-linked glycosylation.

Glycosylation patterns for proteins produced by mammals are described in detail in The Plasma Proteins Structure, Function and Genetic Control, Putnam, F. W., ed., 2nd edition, Vol. 4, Academic Press, New York, 1984, especially pp. 271-315. In this chapter, asparagine-linked oligosaccharides are discussed, including their subdivision into a least three groups referred to as complex, high mannose, and hybrid structures, as well as glycosidically linked oligosaccharides.

In the case of N-linked glycans, there is an amide bond connecting the anomeric carbon (C-1) of a reducing-terminal N-acetylglucosamine (GlcNAc) residue of the oligosaccharide and a nitrogen of an asparagine (Asn) residue of the polypeptide. In animal cells, O-linked glycans are attached via a glycosidic bond between N-acetylgalactosamine (GalNAc), galactose (Gal), fucose, N-acetylglucosamine, or xylose and one of several hydroxyamino acids, most commonly serine (Ser) or threonine (Thr), but also hydroxyproline or hydroxylsine in some cases.

The biosynthetic pathway of O-linked oligosaccharides consists of a step-by-step transfer of single sugar residues from nucleotide sugars by a series of specific glycosyltransferases. The nucleotide sugars which function as the monosaccharide donors are uridine-diphospho-GalNAc (UDP-GalNAc), UDP-GlcNAc, UDP-Gal, guanidine-diphospho-fucose (GDP-Fuc), and cytidine-monophospho-sialic acid (CMP-SA).

In N-linked oligosaccharide synthesis, initiation of N-linked oligosaccharide assembly does not occur directly on the Asn residues of the protein, but involves preassembly of a lipid-linked precursor oligosaccharide which is then transferred to the protein during or very soon after its translation from mRNA. This precursor oligosaccharide (Glc₃Man₉GlcNAc₂) is synthesized while attached via a pyrophosphate bridge to a polyisoprenoid carrier lipid, a dolichol, with the aid of a number of membrane-bound glycosyltransferases. After assembly of the lipid-linked precursor is complete, another membrane-bound enzyme transfers it to sterically accessible Asn residues which occur as part of the sequence -Asn-X-Ser/Thr-.

Glycosylated Asn residues of newly-synthesized glycoproteins transiently carry only one type of oligosaccharide, Glc₃Man₉GlcNAc₂. Processing of this oligosaccharide structure generates the great diversity of structures found on mature glycoproteins.

The processing of N-linked oligosaccharides is accomplished by the sequential action of a number of membrane-bound enzymes and includes removal of the three glucose residues, removal of a variable number of mannose residues, and addition of various sugar residues to the resulting trimmed core.

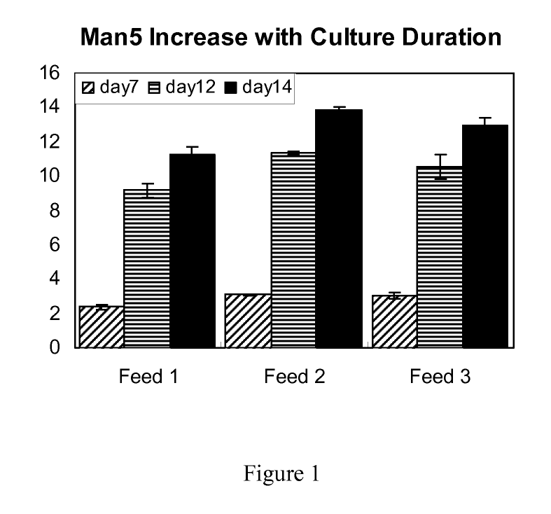

Four of the mannose residues of the Man₉GlcNAc₂ moiety can be removed by α-mannosidase I to generate N-linked Man₅-9GlcNAc₂, all of which are commonly found on vertebrate glycoproteins. As shown in FIG. 1, the Man₅GlcNAc₂ can serve as a substrate for GlcNAc transferase I (GlcNAcT-I), which transfers a β1→2-linked GlcNAc residue from UDP-GlcNAc to the α1→3-linked mannose residue to form GlcNAcMan₅GlcNAc₂, which is further trimmed by α-mannosidase II, which removes two mannose residues to generate a protein-linked oligosaccharide with the composition GlcNAcMan₃GlcNAc₂. This structure is a substrate for GlcNAc transferase II (not shown).

This stage is followed by a complex series of processing steps, including sequential addition of monosaccharides to the oligosaccharide chain by a series of membrane-bound glycosyltransferases, which differ between various cell types. As a result, a diverse family of “complex” oligosaccharides is produced, including various branched, such as biantennary (two branches), triantennary (three branches) or tetraantennary (four branches) structures.

A number of antibody glycoforms have been reported as having a positive impact on antibody effector function, including antibody-dependent cell mediated cytotoxicity (ADCC). This can be of particular benefit in the oncology field, where therapeutic monoclonal antibodies bind to specific antigens on tumor cells and induce an immune response resulting in destruction of the tumor cell. By enhancing the interaction of IgG with killer cells bearing Fc receptors, these therapeutic antibodies can be made more potent.

The present invention discloses methods for producing antibodies having an increased amount of the Man5 glycoform while diminishing the amount of Man7,8,9 relative to what has been previously described. It also describes a method for modulating the amount of the Man5 glycoform produced.

The present invention provides methods for producing antibodies with a variable amount of Man5 using specific cell culturing methods.

In accordance with the methods of the present invention, host-cells are cultured in a medium that allows for the expression of recombinant glycoproteins having high Man5 content. Suitable cell culture procedures and conditions are well known in the art. Host-cells (e.g., CHO cells) may be cultured in a wide variety of formats and culture vessels. For example, host-cells may be cultured in formats designed for large scale or small scale production of glycoproteins. Additionally, host-cells may be cultured adherent to the bottom of culture flasks or dishes, or they may be in suspension in stirred flasks, bioreactors or in roller bottle cultures. In certain embodiments, for production of recombinant glycoproteins in commercially relevant quantities, host-cells may be grown in bioreactors, and preferably bioreactors having a capacity of about 2 liters or more, or about 5 liters or more, or about 10 liters or more, or about 50 liters or more, or about 100 liters or more, or about 500 liters or more, or about 1000 liters or more, or about 1500 liters or more, or about 2000 liters or more.

In certain embodiments, host-cells can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In certain embodiments, host-cells are cultured in shake flasks. In yet other embodiments, host-cells are cultured in a fermentor (e.g., in a fermentation process). Fermentation processes include, but are not limited to, batch, fed-batch and continuous methods of fermentation. The terms “batch process” and “batch fermentation” refer to a closed system in which the composition of media, nutrients, supplemental additives and the like is set at the beginning of the fermentation and not subject to alteration during the fermentation; however, attempts may be made to control such factors as pH and oxygen concentration to prevent excess media acidification and/or microorganism death. The terms “fed-batch process” and “fed-batch” fermentation refer to a batch fermentation with the exception that one or more substrates or supplements are added (e.g., added in increments or continuously) or the cell culture conditions are changed as the fermentation progresses. The terms “continuous process” and “continuous fermentation” refer to a system in which a defined fermentation media is added continuously to a fermentor and an equal amount of used or “conditioned” media is simultaneously removed, for example, for recovery of the desired product (e.g., recombinant glycoprotein). A variety of such processes have been developed and are well-known in the art.

Following the polypeptide production phase, the recombinant glycoprotein of interest can be recovered from the culture medium using techniques which are well established in the art. The glycoprotein of interest preferably is recovered from the culture medium as a secreted polypeptide, although it also may be recovered from host cell lysates.

Various cell culture parameters can affect the mannose content of a recombinant glycoprotein expressed in mammalian cell culture. In particular, it was discovered by way of the present invention that lengthening the duration of time the cells spend in the culture medium results in a higher percentage of Man5 glycoproteins produced. Accordingly, in one embodiment of the present invention, a mammalian cell expressing a recombinant glycoprotein is cultured for a period greater than 12 days to achieve highest possible Man5 levels. In other embodiments the cells are cultured for a period ranging from about 12 days to about 22 days. By contrast, if a low level of Man5 content is desired, glycoprotein expressing cells may be cultured for a shorter cell culture duration of 10 days or shorter. Man5 increases with culture time was observed for all the cell lines tested.

In addition, it was discovered by way of the present invention that the higher the osmolality of the cell culture medium is, the higher the percentage of Man5 glycoproteins produced. Accordingly, in one embodiment of the present invention, osmolality of the cell culture medium is maintained at greater than about 300 mOsm/Kg to increase Man5 content of expressed glycoproteins (e.g., about 300 mOsm/Kg to about 400 mOsm/Kg).

Both basal and feed media osmolality can be adjusted by adding extra NaCl into media to increase Osm. Man5 increases with media osmo, especially basal media osmo, for multiple cell lines. Both feed and basal media osmolity were tested and it was determined that basal media osmolality has a more significant effect towards increasing Man5 content.

For mammalian cell culture, osmolality of the cell culture medium is maintained at greater than about 300 mOsm/Kg, or at greater than about 330 mOsm/Kg, or at greater than about 360 mOsm/Kg, or at greater than about 400 mOsm/Kg, or at between about 300 mOsm/Kg and about 400 mOsm/Kg.

In order to achieve an osmolality in the desired range, the concentration of various constituents in the culture medium can be adjusted. For example, solutes which can be added to the culture medium so as to increase the osmolality thereof include proteins, peptides, amino acids, hydrolyzed animal proteins such as peptones, non-metabolized polymers, vitamins, ions, salts, sugars, metabolites, organic acids, lipids, and the like. It will be appreciated however, that the concentration(s) of other constituents in the culture medium can be modified in order to achieve a desired osmolality. In preferred embodiments, media osmolity can be adjusted by adding different media components, mainly inorganic salts, NaCl, KCl, sodium carbonate, sodium bicarbonate, sodium phosphate, monobasic, bibasic etc.

In other embodiments, osmolality can be adjusted to the aforementioned ranges by adding one or more osmoprotectants to the culture medium. Exemplary osmoprotectants are well known in the art and include, but are not limited to, betaine, glycine, L-threonine, L-proline and derivatives thereof including, but not limited to, glycine betaine, betaine aldehyde.

Osmolality can be measured by any of the means that are well-known in the art and those described herein.

In still other embodiments, the aforementioned parameters affecting osmolality can be combined with manipulating the temperature and duration of time which the cells are cultured to modulate (e.g., increase) mannose-content. Accordingly, it should be understood that the various cell culturing parameters described herein can be adjusted alone or in combination to modulate the mannose-content of recombinant glycoproteins.

Mn is the cofactor of GnT1, which converts Man5 into a hybrid glycoform. Adding or reducing Mn in the cell culture media may impact the GnT1 activity. In the experiments leading up to the present invention, it was also demonstrated that the presence of MnCl₂ in the culture medium contributes to a reduction in the Man5 content of glycoproteins. Accordingly, in one embodiment, the invention employs a cell culture medium having a MnCl₂ concentration of about 0.25 μM or less, or preferably using a cell culture medium absent of MnCl₂.

As discussed above, the MnCl₂ concentration of the cell culture medium alone may be controlled or it may be controlled in combination with one or more of the other factors described herein which affect Man5 levels.

In various embodiments of the present invention, the above cell culturing methods may be used alone or in combination to achieve maximal Man5 levels.

In another aspect of the present invention, the above cell culturing methods may be used in combination with other methods to achieve maximal Man5 levels.

In one embodiment, the cell culturing methods of the present invention may be used in conjunction with a mammalian cell lacking GlcNAc Transferase I activity. In a particular embodiment this would include a method for producing antibodies with a variable amount of Man5 using RNA interference (RNAi) knockdown in conjunction with the optimized cell culturing conditions.

RNA interference (RNAi) is a method for regulating gene expression. RNA molecules can bind to single-stranded mRNA molecules with a complementary sequence and repress translation of particular genes. The RNA can be introduced exogenously (small interfering RNA, or siRNA), or endogenously by RNA producing genes (micro RNA, or miRNA). For example, double-stranded RNA complementary to GlcNAc Transferase I can decrease the amount of this glycosyltransferase expressed in an antibody expressing cell line, resulting in an increased level of the Man5 glycoform in the antibody produced. Unlike in gene knockouts, where the level of expression of the targeted gene is reduced to zero, by using different fragments of the particular gene, the amount of inhibition can vary, and a particular fragment may be employed to produce an optimal amount of the desired glycoform. An optimal level can be determined by methods well known in the art, including in vivo and in vitro assays for Fc receptor binding, effector function including ADCC, efficacy, and toxicity. The use of the RNAi knockdown approach, rather than a complete knockout, allows the fine tuning of the amount of Man5 glycan to an optimal level, which may be of great benefit, if the production of antibodies bearing less than 100% Man5 glycans is desirable.

The α-1,2 mannosidase activity can be enhanced in a variety of ways. For example, α-1,2 mannosidase activity can be enhanced by providing additional copies of the α-mannosidase I present in the recombinant host cell used for antibody production. Alternatively, mannosidases may be used post expression in vitro to trim Man6,7,8,9 to Man5.

Other compounds that are capable of inhibiting alpha mannosidases would be applicable in the present invention, including inhibitors that block only alpha 1,2 mannosidases as well as inhibitors that, in addition, are capable of inhibiting other mannosidases as well. Thiosugar derivatives that are more potent than kifunensine have been described (Sivapriya et al, Bioorg Med Chem (2007) 15 (17): 5659-65). Other alpha mannosidase inhibitors include, but are not limited to, iminocyclitols (Butters et al, Glycoconj J. (2009) epub), 1-deoxymannojirimycin (Bischoff et al, J. Biol. Chem. (1986) 261:4766-4774), kifunensine analogues (Hering et al, J Org Chem (2005) 70: 9892-904), and D-Mannonolactam Amidrazone (Pan et al, J Biol Chem (1992) 267: 8313-8318).

The following examples are offered for illustrative purposes only, and are not intended to limit the scope of the present invention in any way.

All patent and literature references cited in the present specification are hereby incorporated by reference in their entirety.

### Example 1

### Manipulating Cell Culture Conditions to Increase Man5 Level

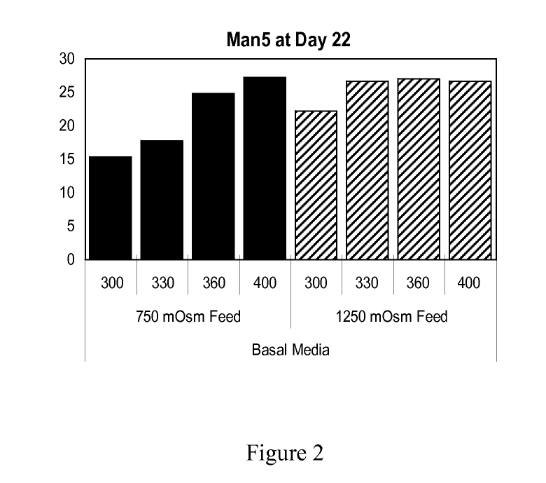

The use of optimized cell culture parameters can increase the amount of Man5 obtained. Longer culture duration and increased osmolality media have been found to be beneficial with another antibody evaluated, and results by others (US patent application US2007/0190057-A1 FIG. 2, FIG. 4) have also shown that increasing osmolality can increase the proportion of antibodies with high mannose glycoforms.

Materials And Methods

Cell Line and Culture Conditions

FGF19 is a CHO derived cell line from dihydrofolate reductase minus (dhfr−) CHO DP-12 host. This cell line was adapted for growth in a serum-free medium, amplified using methotrexate selection, developed by cell line development group.

Shake flask cultures were seeded at 1.0×10⁶ cells·ml⁻¹ in a 125 mL shake flasks with 40 mL working volume (VWR 125 mL Polycarbonate, Erlenmeyer/Shaker Culture Flask with PTFE 0.22 umm vented cap). In a humidified incubator Kuhner ShakerX Climo-Shaker 1SF1-X, the cells were grown initially at 37° C. then shifted to lower temperature at 35° C. on day 2 with constant 150 rpm agitation, 5% CO2, and 85% humidity for a duration of 14 to 22 days in fed-batch conditions.

Stock proprietary basal media and batch feed were used for this experiment. Osmolalities of both basal and feed were adjusted to meet desired levels during media preparation. One lot of basal media was split into four solutions and adjusted to have corresponding osmolalities: 300, 330, 360 & 400 mOsm. Similarly, one lot of feed media was split into two solutions to have 750 and 1250 mOsm. Osmolality adjustments were achieved with appropriate additions of NaCl (Gibco Invitrogen). For all media prepared, NaCl powder was added, dissolved in solution, and then filtered prior to usage. To calculate the amount of NaCl required in weight, number of particles that dissociates and its molecular weight were integrated into the formula. [NaCl grams=(desired osmo−initial osmo)×2/58.4×Batch size in mL]. Osmolality was verified with a micro osmometer Model 3300 by Advanced Instruments, Inc upon preparation. 1 uM stock solution of MnCl₂ (Sigma) was prepared then filtered. All solutions were filtered with VWR 0.2 uM bottle top vacuum filters.

The cultures were grown in respective shake flasks containing corresponding basal media osmolalities of 300, 330, 360 & 400 mOsm. Fed three times on days 3, 6 & 9, each feed volume was 10% (4 mL) of the initial working volume (40 mL). Each basal osmolality condition had two cases. One case tested with 750 mOsm feed while the other condition was tested with 1250 mOsm feed.

In another set of shake flasks, conditions containing MnCl₂ were carried out only for 14 days with one or three feeding strategy, same feed volume of 10% added regardless of feeding frequency. Prepared MnCl₂ was titrated in trace amounts to cell cultures fed only with 750 mOsm feed media. In one experiment, three feeds were employed with MnCl₂ stock solution directly added to cell culture shake flasks with final concentration of 0.25, 0.50 and 1.0 uM. In another experiment, one feed was employed with final MnCl₂ concentration of 1.0, 2.0 and 3.0 uM.

Cell, Metabolite and Osmolality Analysis

Samples were taken from the shake flask on days 3, 7, 10, 12, 14, 18 and 22. Cell counts, done in Beckman Coulter Vi-CELL Viability Analyzer, provided viable cell counts (VCC), viability and cell size. Metabolic analysis, done in Nova Biomedical Bioprofile 400 Analyzer, measured lactate, ammonia, glucose, etc. Osmolality readings were done in Advanced Instruments Advanced Instrument micro osmometer Model 3300. Collected supernatant samples were stored at −80 C followed by removal of cells through centrifugation at 2000 rpm for 10 minutes. Frozen samples were thawed and collectively submitted for glycan analysis.

CE-Glycan Assay

To determine the actual Man5 level of the antibodies collected in HCCF, capillary electrophoresis, referred to as “CE-glycan”, was selected to be the standard method to measure released glycans from the antibody. Briefly, collected supernatant samples on days 7, 12, 14, 18 & 22 were purified using protein A column, using automated sampling platform, OASIS. After desalting with Microcon 30 concentrator and buffer exchanged with Peptide-N-Glycosidase F (PNGase F), the samples were spin down multiple times at different speed and duration. The proteins were digested with enzyme PNGase F overnight in a water bath at 37 C. Released asparagine linked carbohydrates were recovered by centrifugation followed by precipitation at 95° C. Oligosaccharide pellets were released by low heat drying in a centrifugal drying evaporator (CVE) then labeled with APTS working labeling reagent. The labeled glycans were then analyzed using capillary electrophoresis against APTS-labeled glycan standards with specific elution profile. 1M sodium cyanoborohyde in THF was added to dissolved pellets then incubated at a 55° C. prior to injection to capillary electrophoresis (CE) with fluorescence detection (Beckman P/ACE MDQ series and PA800). The details of the assay can be found on the Beckman Coulter website. The relative amounts of glycans with major and minor carbohydrate moieties such as G0, G1, G2, and Man5 were determined.

Results

FIG. 1 is an example of a production run of the antibody evaluated, which clearly shows that a large amount of Man5 antibodies were produced toward the end of the 14 days culture. In addition, increased NaCl (or osmolality) concentration in basal media was also tested with respect to level of Man5. As shown in FIG. 2, increasing basal osmolality from 300 to 400 mOsm can further increase Man5 content. However, the addition of high osmolality nutrient supplement solution does not enhance the Man5 level beyond the benefit of the high osmolality basal media (data not shown). The high osmolality and longer culture duration effect can be used in combination in order to increase the Man5 level for other molecules. Due to these findings, an experiment was designed to test these conditions with the cell line generating ocrelizumab and the top 5 GnT-I knockdown stable clones of ocrelizumab described in the previous section.

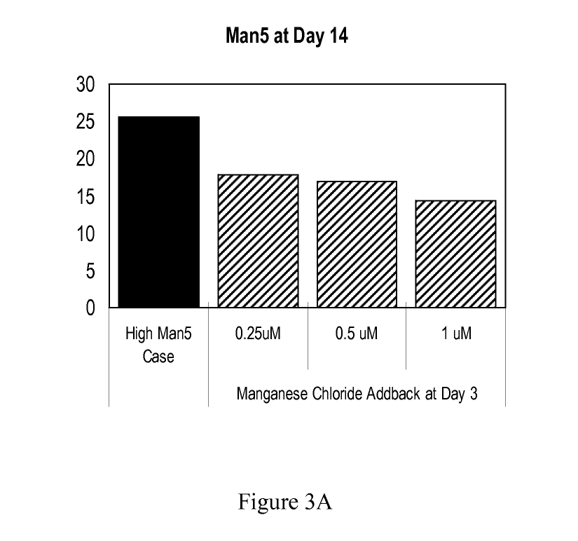

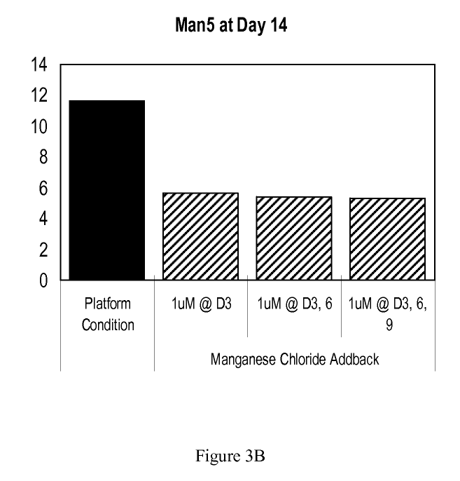

In addition to the effect caused by osmolality and culture duration, the addition of manganese has been shown to reduce the Man5 level when a small amount of manganese chloride was fed into the culture. FIG. 3A summarizes the results from adding different amount of Manganese on Day 3, 0.25, 0.5, 1 uM. FIG. 3B summarizes the results of a 14 day production run with the same antibody, where 1 μM of manganese chloride was fed on either day 3, day 3 & 6, or day 3, 6, & 9. The results show there is a dose response when adding Manganese on day 3, but further adding on day 6, and 9 cannot further reduced Man5 level. In all cases, the Man5 level was decreased by 50% compared to the control. To increase the Man5 level, conditions which lower manganese concentration would be expected to be beneficial.

In general, Man5 level increases as culture duration increases for all conditions. High osmolality in basal media appears to have the strongest effect in enhancing the Man5 level, and the absence of manganese has a slight benefit as compared to the control. By extending the production culture from 14 days to 21 days and the usage of high osmolality basal media, the Man5 level can be increased up to 2-fold. Therefore, by manipulating cell culture conditions, the Man5 level can be further enhanced.

Throughout the foregoing description the invention has been discussed with reference to certain embodiments, but it is not so limited. Indeed, various modifications of the invention in addition to those shown and described herein will become apparent to those skilled in the art from the foregoing description and fall within the scope of the appended claims.

1-39. (canceled)

40. A method for producing an antibody or a fragment thereof, or an immunoadhesin or a fragment thereof, bearing Man5 glycans, comprising culturing a cell line engineered to express an antibody or a fragment thereof, or an immunoadhesin or a fragment thereof, comprising
(a) culturing for a duration of time greater than 12 days;
(b) using culture medium having a basal media osmolality of about 300 mOsm/Kg or greater; or
(c) using culture medium having a Manganese concentration of about 0.25 micromolar or less;
wherein the culturing conditions lead to the accumulation of Man5 glycoproteins.

41. The method of claim 1 wherein the duration of cell culture is between 12 and 22 days.

42. The method of claim 1 wherein the osmolality of the basal culture medium is between about 300 and about 400 mOsm/Kg.

43. The method of claim 1 wherein Manganese is removed from the culture medium.

44. The method of any of claims 1 wherein the cell line is a mammalian cell line.

45. The method of any of claims 1 wherein the mammalian cell line is a Chinese Hamster Ovary (CHO) cell line.

46. The method of any of claims 1 wherein the production phase is a batch or fed batch culture phase.

47. The method of any of claims 1 further comprising the step of isolating said antibody, or a fragment thereof, or immunoadhesin, or a fragment thereof.

48. The method of claim any of claims 1 wherein the antibody or fragment thereof, or the immunoadhesin or fragment thereof, bear 10% or greater Man5 glycans.

49. The method of any of claims 1 wherein the antibody or fragment thereof, or the immunoadhesin or fragment thereof, bear 20% or greater Man5 glycans.

50. The method of any of claims 1 wherein the antibody or fragment thereof, or the immunoadhesin or fragment thereof, bear 30% or greater Man5 glycans.

51. The method of any of claims 1, wherein the antibody or antibody fragment binds to an antigen selected from the group consisting of CD3, CD4, CD8, CD19, CD20, CD22, CD34, CD40, EGF receptor (EGFR, HER1, ErbB1), HER2 (ErbB2), HER3 (ErbB3), HER4 (ErbB4), macrophage receptor (CRIg), tumor necrosis factors, TRAIL/Apo-2, LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DR5, EGFL7, neuropilins and receptors thereof, VEGF-C, ephrins and receptors thereof, netrins and receptors thereof, slit and receptors thereof, sema and receptors thereof, semaphorins and receptors thereof, robo and receptors thereof, and anti-M1.

52. The method of claim 14 wherein said antibody is chimeric or humanized.

53. The method of claim 15 wherein the chimeric antibody is an anti-CD20 antibody.

54. The method of claim 16 wherein the anti-CD20 antibody is rituximab or ocrelizumab.

55. The method of claim 15 wherein the humanized antibody is an anti-HER2, anti-HER1, anti-VEGF or anti-IgE antibody.

56. The method of claim 18 wherein the anti-HER2 antibody is trastuzumab or pertuzumab.

57. The method of claim 18 wherein the anti-VEGF antibody is bevacizumab, or ranibizumab.

58. The method of claim 18 wherein the anti-IgE antibody is omalizumab.

59. The method of claim 14 wherein the antibody fragment is selected from the group consisting of complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide.

60. The method of claim 1, wherein the culturing conditions comprise the combination of culturing for a duration of time greater than 12 days and using a culture medium having a basal media osmolality of about 300 mOsm/Kg or greater.

61. The method of claim 21, further comprising the use of a culture medium having a Manganese concentration of about 0.25 micromolar or less.

62. The method of claim 1, wherein the culturing conditions comprise the combination of culturing for a duration of time greater than 12 days and using a culture medium having a Manganese concentration of about 0.25 micromolar or less.

63. The method of claim 1, wherein the culturing conditions comprise combining the use of a culture medium having a basal media osmolality of about 300 mOsm/Kg or greater and the use of a culture medium having a Manganese concentration of about 0.25 micromolar or less.

64. The method of claim 1, further comprising the use of a mammalian cell lacking or with substantially diminished GlcNAc Transferase I activity.

65. A method for recombinant production of an antibody, an immunoadhesin, or a fragment thereof with about 10% to 30% Man5 glycans in the carbohydrate structure thereof, comprising
expressing nucleic acid encoding said antibody or antibody fragment in a mammalian cell line, wherein said fragment comprises at least one glycosylation site,
culturing said cell line under conditions suitable for the accumulation of Man5 glycoproteins comprising culturing for a duration of time greater than 12 days, using culture medium having a basal media osmolality of about 300 mOsm/Kg or greater, or culture medium having a Manganese concentration of about 0.25 micromolar or less, and
isolating said antibody or a fragment thereof, or an immunoadhesin or a fragment thereof, bearing predominantly Man5 glycans.

66. The method of claim 26, wherein the antibody or antibody fragment binds to an antigen selected from the group consisting of CD3, CD4, CD8, CD19, CD20, CD22, CD34, CD40, EGF receptor (EGFR, HER1, ErbB1), HER2 (ErbB2), HER3 (ErbB3), HER4 (ErbB4), LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DR5, EGFL7, neuropolins and receptors thereof, VEGF-C, ephrins and receptors thereof, netrins and receptors thereof, slit and receptors thereof, sema and receptors thereof, semaphorins and receptors thereof, robo and receptors thereof, and anti-M1.

67. The method of claim 31 wherein said antibody is chimeric or humanized.

68. The method of claim 31 wherein the antibody fragment is selected from the group consisting of complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide.
